(19) United States
(12) Patent Application Publication
(10) Pub. No.: US 2008/0081048 A1
(43) Pub. Date: Apr. 3, 2008

(54) Production of modified neurotoxins

(76) Inventors: Laurence Raymond, Plantation, FL (US); Paul Reid, Plantation, FL (US)

Correspondence Address: Robert J. Van Der Wall;Gables One Tower, Suite 1275, 1320 South Dixie Highway, Coral Gables, FL, 33146, US

(21) Appl. No.: 11/540,862

(22) Filed: Oct. 2, 2006

(51) Int. Cl. A61K 39/08; C07K 14/195

(52) U.S. Cl. 424/239.1; 530/350

(57) ABSTRACT

The invention provides improved methods of making compositions comprising detoxified neurotropically active modified neurotoxins derived from snake venom, and compositions made using these methods. The compositions are useful for the treatment of a wide variety of neurological and viral diseases.

### CROSS REFERENCE TO RELATED APPLICATION

The present application cross-references a provisional patent application Ser. No. 60/351,462 filed Jan. 28, 2002.

### BACKGROUND OF THE INVENTION

1. Field of the Invention

The present invention relates to an improvement in the process of production of modified neurotoxins used for treatment of neurological and viral diseases and especially to the treatment of heretofore intractable diseases such as Rabies, Myasthenia Gravis, HIV, Dementia, Muscular Dystrophy, Multiple Sclerosis and Amyotrophic Lateral Sclerosis through modulation or blockade of the nicotinic acetylcholine receptor. The improved method permits the more rapid production and improved quality of such therapeutic peptides relative to prior published methods.

2. Description of the Prior Art

Sanders et al. had commenced investigating the application of modified venoms to the treatment of ALS in 1953 having employed poliomyelitis infection in monkeys as a model. Others antiviral studies had reported inhibition of pseudorabies (a herpesvirus) and Semliki Forest virus (alpha-virus). See Sanders' U.S. Pat. Nos. 3,888,977, 4,126,676, and 4,162,303. Sanders justified the pursuit of this line of research through reference to the studies of Lamb and Hunter (1904) though it is believed that the original idea was postulated by Haast. See Haast U.S. Pat. Nos. 4,741,902 and 5,723,477. The studies of Lamb and Hunter (Lancet 1:20, 1904) showed by histopathologic experiments with primates killed by neurotoxic Indian cobra venom that essentially all of the motor nerve cells in the central nervous system were involved by this venom. A basis of Sanders' invention was the discovery that such neurotropic snake venom, in an essentially non-toxic state, also could reach that same broad spectrum of motor nerve cells and block or interfere with invading pathogenic bacteria, viruses or proteins with potentially deleterious functions. Thus, the snake venom used in producing the composition was a neurotoxic venom, i.e. causing death through neuromuscular blockade. As the dosages of venom required to block the nerve cell receptors would have been far more than sufficient to quickly kill the patient, it was imperative that the venom be detoxified. The detoxified but undenatured venom was referred to as being neurotropic. The venom was preferably detoxified in the mildest and most gentle manner. While various detoxification procedures were known then to the art, such as treatment with formaldehyde, fluorescein dyes, ultraviolet light, ozone or heat, it was preferred that gentle oxygenation at relatively low temperatures be practiced, although the particular detoxification procedure was not defined as critical. Sanders employed a modified Boquet detoxification procedure using hydrogen peroxide, outlined below. The acceptability of any particular detoxification procedure was primarily the determination of a lack of toxicity; anti-viral potency was tested by the classical Semliki Forest virus test, as taught by Sanders, U.S. Pat. No. 4,162,303. As taught by Sanders, the composition of the detoxification solution was as indicated below, with regard to the components active in detoxification.

DETOXIFICATION SOLUTION COMPONENTS

| Lyophilized cobra sp. venom | 40 | g |
|---|---|---|
| CuSO₄ (1% solution) | 2 | mL |
| H₂O₂ (30%) | 80 | mL |
| Phosphate buffered saline*, pH 7.6, q.s. | 4000 | mL |
| Antifoam | (trace) | |
| Formalin (39%)** | 8 | mL |

(*Sorenson's buffer; **substance later omitted)

The end concentration of cobra venom in the detoxification solution was 10 mg/mL; the end concentration of H₂O₂ was 196 mM. The theoretical level of CuSO₄, based upon complete solubilization, which acts as a catalyst for the action of H₂O₂ upon proteins, was 31.3 uM. The solution was suggested to be incubated at a temperature range of between 15° C. to 40° C., with the preferred range being 20° C. to 40° C. with, or without, agitation.

The term of incubation to accomplish detoxification is up to 30 days, especially 6 to 16 days, dependent upon the venom type and temperature of incubation. Shorter or longer periods of time were indicated as acceptable as long as the bio-assay for toxicity in mice and the Semliki-virus plaque assay were acceptable.

Literature references of interest are: Hinmann C. L., Stevens-Truss R., Schwarz C., Hudson R. A. Immunopharmacol Immunotoxicol. (1999) August;21(3):483-506., Hudson R A, Montgomery I N and Rauch H C. Mol Immunol. (1983) February;20(2):229-32; Lamb, G and Hunter, W. K, The Lancet, 1: 20-22; Marx, A., Kirckner, T., Hoppe, F., O'Connor, R., Schalke, B., Tzartos, S. and Muller-Hermelink, H. K., Amer. J. Path, (1989) 134, No. 4, 865-75; Miller, K., Miller, G. G., Sanders, M. And Fellowes, O. N., Biophys et Biophysica Acta 496:192-196) (1977); Sanders, M., Soret, M. G. and Akin, B. A.; Ann. N. Y. Acad. Sci. 53: 1-12 (1953); Sanders, M., Soret, G., and Akin, B. A.; J. Path. Bacteriol. 68:267-271 (1954); Sanders M. And Fellows O.; Cancer Cytology 15:34-40(1975) and in Excerpta Medica International; Congress Series No. 334 containing abstracts of papers presented at the III International Congress of Muscle Diseases, Newcastle on Tyne, September 1974; Sanders M., Fellowes O. N. and Lenox A. C.; In: Toxins: Animal, Plant and Microbial, Proceedings of the fifth international symposium; P. Rosenberg, editor, Pergamon Press, New York 1978, p. 481;

### SUMMARY OF THE INVENTION

In accordance with a principal aspect of the invention, there is provided an improved method of drug production by modification of the procedure by Sanders, in which the native neurotoxin or venom is detoxified by controlled oxygenation. The present composition may also be produced from any venom or venom component that acts, essentially, as a neurotoxin, as opposed to, essentially, a hematoxin.

The present invention accomplishes the above-stated objectives, as well as others, as may be determined by a fair reading and interpretation of the entire specification.

### DETAILED DESCRIPTION OF THE INVENTION

As required, detailed embodiments of the present invention are disclosed herein; however, it is to be understood that the disclosed embodiments are merely exemplary of the invention which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

Using Sander's patented formula, it was determined by the present inventors that the incubation of venom mixtures from Naja naja (NNV), Naja Kaouthia (NKV)and the neurotoxic component from NKV, known as cobratoxin, at a temperature of 37+1° C., resulted in a detoxified product in 9 to 15 days. Detoxification was determined by the mouse toxicity assay through the survival of a 0.5 mL intraperitoneal (IP) injection of the detoxification solution. Incubation of the reaction mixtures at lower temperatures, i.e., room temperature (22-23° C.) resulted in an increase in the time to detoxification in comparison to incubation at 37° C. Additionally, as indicated by Sanders, different venoms required variable periods of incubation, as demonstrated by Naja nivea venom (N), which requires 30 days at 37° C. Similarly, as indicated above, when cobratoxin, the neurotoxic component of NKV, rather than whole venom was detoxified, a period of 9-14 days was required, coinciding with the detoxifying period for the source whole venom, all other conditions remaining constant.

In all the above indicated cases, copper sulphate (CuSO₄) was solubilized in Sorenson's buffer, as inferred from Sanders Pat. No. 3,888,977 patent where Sorenson's buffer was the only solvent noted. However, when water was used solely as the solvent for CuSO₄, the resulting detoxification occurred in an abbreviated period in comparison to the time frame required when Sorenson's buffer was employed as the solvent for CuSO₄. The time frames for the detoxification of several venoms and cobratoxin (CT) using either water or Sorenson's buffer as the solvent for the solubilization of CuSO₄ are indicated in Example 1 below.

In general, considering the time periods from the table above, the period of time required to detoxify cobratoxin using CuSO₄ solubilized in water at 37° C. requires 21.4% to 42% of the time to detoxify using Sorenson's as a solvent for CuSO₄. The effect is similar for detoxifying Naja naja venom at 37° C., where 26% of the time is required for detoxification when water is the Cu++ solvent. This time period for detoxification using CuSO₄/water is increased to 59% of the time required for detoxification using CuSO₄/Sorenson's, when the temperature is reduced to 22.7° C. (73° F.; room temperature). The detoxification of Naja kaouthia venom with CUSO₄/water requires 35% of the time required compared to the use of CUSO₄/Sorenson's and Naja nivea (flava) detoxification time is decreased by 60% in contrast to the use of CuSO₄/Sorenson's. This data is presented in Table 2.

There is a noticable difference in the appearance of a 1% CuSO₄ solution when water is used as a solvent as versus Sorenson's. In water the solution is clear; in Sorensen's buffer the solution is cloudy. Thus the apparent solubility is greater in water than in Sorenson's. The alteration of apparent solubility in Sorenson's buffer, which is a phosphate buffer, may be the production of cupric phosphate which is insoluble in water. Therefore, switching the solubilization of CuSO₄ into water, rather than in Sorenson's buffer, results in a significant decrease (Students t-Test; P=0.001) in the time to produce detoxification, regardless of the temperature of incubation or the type of venom, or component.

A further improvement on the Sander's formulation can be achieved through the use of bovine catalase linked to agarose. This provides three advantages. In an era where there are concerns over the use of an animal-derived material in biological products Sanders use of soluble catalase is no longer acceptable. Soluble catalase is rarely pure in composition and often heavily contaminated with bacterial endotoxins. Bovine catalase is not an active part of the drug matrix and it is preferable that it be removed after it has served it purpose of degrading hydrogen peroxide. Catalase linked to agarose also cross-links contaminating proteins, possibly prions, and it can be washed to remove contaminating viruses and endotoxin prior to use in the removal of hydrogen peroxide. Following the completion of the reaction the agarose-linked catalase settles out of solution and can be removed from the product. This greatly improves the resulting quality of the composition for parenteral administration.

Sanders method (U.S. Pat. No. 3,888,977) also described the requirement for dialysing the resulting composition, a step that can be eliminated for two reasons. Firstly, the levels of copper do not represent a hazard to the host's health and therefore does not need to be removed. It was also discovered that the dialysis step could permit the loss of detoxified neurotoxins from reacted preparations—a component critical to the activity and potency of the product. These improvements permit a greater process speed and increase the potential manufacturing output without affecting the final quality of the product. In fact, product made using the water solubilization of copper sulphate appeared to demonstrate a slightly increased potency.

### EXAMPLE 1

Variations in Detoxification Time Due to the Use of Different Solvents for CuSO₄ are shown in Table 1.

TABLE 1

| | Venom (V) or | | Days to |
|---|---|---|---|
| Lot ID | Toxin (T) | CuSO₄ solvent | Detoxify |
| COBRATOXIN | | | |
| 20050921mCT 1 | CT | Water | 3 |
| 20060517mCT | CT | Water | 4 |
| | | AVERAGE | 3.5 |
| 20050921mCT 2 | CT | Sorenson's | 14 |
| 20051110mCT | CT | Sorenson's | 9 |
| 20060215mCT | CT | Sorenson's | 10 |
| | | AVERAGE | 11 |
| VENOM | | | |
| T63 | NNV | Sorenson's | 15 |
| T42D | NKV | Sorenson's | 15 |
| 20051110mNKV | NKV | Sorenson's | 12 |
| 20051110mNKVnCT | NKV | Sorenson's | 12 |
| 20060321mN | N | Sorenson's | 30 |
| | | AVERAGE | 16.8 |
| T62 | NNV | water | 4 |
| T42A | NKV | water | 6 |
| T42B | NKV | water | 6 |
| T42C | NKV | water | 4 |
| 20060327mN | N | water | 12 |
| | | AVERAGE | 6.4 |
| T64 | NNV | water | 16 (RT) |
| T65 | NNV | Sorenson's | 27 (RT) |

Key:
CT: Cobratoxin;
NNV: Naja Naja venom;
N:: Naja nivea;
NKV: Naja kaouthia venom;
RT: incubation at room temperature

### EXAMPLE 2

The Percent of Time to Detoxify using water (W) versus Sorenson's buffer (S) as a solvent for CuSO₄ is shown in Table 2.

TABLE 2

| | Numerator/ | |
|---|---|---|
| Lot Description | denominator | % |
| 20050921mCT (W v S) | (W) 3/(S) 14 | 21.4% |
| 20060517mCT (W) v | (W) 4/(S) 9.5 | 42% |
| 20051110mCT (S) and | | |
| 20060215mCT (S) (averaged time) | | |
| T62/T63 - NNV @ 37 (W v S) | (W) 4.6/(S) 15 | 26% |
| T64/T65 @ RT (W v S) | (W) 16/(S) 27 | 59% |
| T42 A, B, C (W) (averaged time) v | (w) 4.6/(s) 13 | 35% |
| 20051110NKV (S); 2005NKV (S) and | | |
| T42 (NKV) (S) (averaged time) | | |
| 20060327mN (W) V 20060321mN (S) | (w) 12/(s) 30 | 40% |

(W) = water solvent for CuSO₄;
(S) = Sorenson's solvent for CUSO₄

### EXAMPLE 3

A comparison of the efficiency of hydrogen peroxide removal was undertaken using soluble catalase and agarose-linked catalase. The utility of agarose-linked catalase was being examined as it could be removed following the reduction H₂O₂ thereby eliminating a potential source of impurities in the drug substance. To a standard reaction solution of cobra venom or cobratoxin either catalase linked to agarose, was added to 100 IU/mL reaction solution, or soluble catalase derived from aggregated catalase was added to 2670 IU/ml. Agarose linked catalase appears to have a higher reactivity compared to soluble material possibly due to enhance stability as a consequence of cross-linking. In each case the resulting reaction solution was incubated at room temperature for ˜24 hours. Prior to terminating incubation, the level of hydrogen peroxide was determined using the PeroxiDetect Kit obtained from Sigma Chemicals (St. Louis, Mo.; USA). The initial concentration of hydrogen peroxide was 196 mM in all cases. A reduction in the concentration of H₂O₂ below a level of 80 uM is considered to meet the requirements set for an acceptable level of hydrogen peroxide. Routinely the reaction is incubated for 24 hours at which time it is tested.

Data for the lot of drug substance, the type of catalase (soluble or catalase linked to agarose), the hours of incubation in the presence of catalase and the end concentration of peroxide are presented in the Table below.

| | Soluble | Catalase- | Hours | end |
|---|---|---|---|---|
| Lot | catalase | Agarose | Incubation | [H₂O₂] |
| 20051110mCT* | X | | 48 | 31 μM |
| 20051110mNNKV | X | | 24 | 28 μM |
| 20051110mNNKV no CT | X | | 24 | 57 μM |
| 20060215mCT | | X | 22 | 49 μM |
| 20051009mNNKV | | X | 23 | 51 μM |
| 20050921mCT #1 | | X | 23 | 59 μM |
| 20050921mCT #2 | | X | 24 | 50 μM |

*extended incubation and presumed maximum H₂O₂ reduction

As indicated in the Table above, the time frame to decrease the initial 196 mM concentration of H₂O₂ to less than 80 uM is essentially the same for the two preparations: ˜24 hours.

While the invention has been described, and disclosed in various terms or certain embodiments or modifications which it has assumed in practice, the scope of the invention is not intended to be, nor should it be deemed to be, limited thereby and such other modifications or embodiments as may be suggested by the teachings herein are particularly reserved especially as they fall within the breadth and scope of the appended claims.

1. A method of producing a composition including detoxified and neurotropically active modified alpha-neurotoxin suitable for administering to a subject, comprising:
(a) providing a composition including alpha-neurotoxin derived from a whole venom of a snake, or an active fraction thereof;
oxygenating the composition under conditions of a pH of above 7 and at a temperature of 15-40° C.
(c) employing copper sulphate to catalyze the oxygenation;
(d) incubating under said conditions for a period of time sufficient to detoxify the alpha-neurotoxin in the composition;
(e) adding an active catalase linked to a carrier;
(f) removing said catalase from the composition; and
(g) optionally dialyzing the composition.

2. The method of claim 1, wherein the copper sulphate is solubilized in water.

3. The method of claim 1, wherein the step (g) is omitted to prevent loss of detoxified neurotoxins.

4. The method of claim 1, wherein the catalase is bovine catalase linked to agarose.

5. The method of claim 1, wherein the oxygenating is carried out at a temperature of 37° C.

6. The method of claim 2, wherein the length of time required for the detoxification of the composition is reduced, relative to the time required when the copper sulphate is solubilized in a buffer.

7. The method of claim 1, wherein the composition is derived from the venom of a snake selected from the group consisting of Bungarus, Naja naja, Naja nivea, and Naja kaouthia.

8. A composition comprising detoxified and neurotropically active modified alphaneurotoxin suitable for administering to a subject, produced by the method of claim 1.
